(19) United States
(12) Patent Application Publication
(10) Pub. No.: US 2016/0174554 A1
(43) Pub. Date: Jun. 23, 2016

(54) COMPOSTION AND METHOD FOR THE CONTROL OF PHYTO-PARASITIC NEMATODES

(71) Applicant: Derivados Biotecnologicos Sac Derbiotec, Lima (PE)

(72) Inventor: Herbert Telge Noriega, Lima (PE)

(21) Appl. No.: 14/697,736

(22) Filed: Apr. 28, 2015

(30) Foreign Application Priority Data
Dec. 18, 2014 (PE) 002480-2014

(51) Int. Cl. A01N 37/40; C07C 69/90; A01N 43/16; A01M 25/00; A01M 7/00; A01M 9/00; A01M 11/00; A01N 63/04; A01N 65/00

(52) U.S. Cl. CPC A01N 37/40; A01N 63/04; C07C 69/90; A01N 43/16; A01N 65/00; A01M 7/0003; A01M 9/00; A01M 11/00; A01M 25/006

(57) ABSTRACT

The present invention provides a nematicide composition and a method using said composition, for controlling plant pathogenic nematodes. Said composition comprises as active principles, oligomers derived from monomers of gallic acid, quinic acid, caffeic acid and methylated derivatives thereof; also contains hydrocolloids, carbohydrates, water-soluble proteins and agriculturally acceptable excipients. The method for controlling nematodes comprises applying the nematicide composition on the soil of infected crops at appropriate doses and time intervals.

### BACKGROUND

1. Field of the Invention

The present invention is related to the agriculture technical field, specifically related with pesticides, and particularly provides a composition and a method that uses said composition for the elimination of pathogenic nematodes present in plants.

2. Description of Related Art

Parasitic plant nematodes are a major problem for agriculture around the world. More than 4.100 species of plant-parasitic nematodes that affect different types of crops in different territories and soil conditions, have been identified (Nicol J M, Turner S J, Coyne D L, den Nijs L Hockland, Tahna Maafi Z. Current Nematode Threats to World Agriculture.) Chapter 2. Genomics and Molecular Genetics of Plant-Nematode Interactions. 2011). The most harmful plant nematodes include species as Meloidogyne incognita, Rotylenchulus reniformis, genres and Xiphinema Tylenchulus, among others. The plant-parasitic nematodes are the most widely distributed pest and often one of the most difficult and costly to eradicate. It is estimated that the losses of crops caused by nematodes corresponds to a 14.6% and an 8.8% in undeveloped countries and developing countries, respectively (Nicol J M, Turner S J, Coyne D L, den Nijs L Hockland, Tahna Maafi Z. Current Nematode Threats to World Agriculture.) Chapter 2. Genomics and Molecular Genetics of Plant-Nematode Interactions. 2011).

Different strategies of elimination and control of plant-parasitic nematodes have been developed. The first approaches with nematicides was the use of non phytotoxic chemical compounds as fumigants (methyl bromide, 1.3dichloride pentane, chlorpricrine), (thionazine, terbufs, phosphoreditioic acid) organophosphates and carbamates (aldicarb, carbofurane, oxamil). The disadvantage of the use of these nematicides, is that they are highly hazardous to human health, causing poisoning and potential death. Some countries have already banned its use, due to the presence of these toxins in groundwaters and drinking water, and toxic traces in plants and fruits. As a result, these compounds should be applied with caution and for its application they require a specialized staff and specialized machinery in order to avoid accidents (Gowen S R., Chemical control of nematodes: efficiency and side-effects.) FAO Plant Production and Protection Paper—144. Rome 1997). Because of this, the development of new nematicides has made important improvements toward obtaining natural and/or biological products for this purpose.

Within the first mentioned, different plant extracts have been isolated for the preparation of biocides. Some examples are those disclosed in US Patent Application 2012/0128648, in which a nematicide composition is mentioned, which is extracted from a certain species of Eucalyptus. Moreover, Chinese patent CN102246829 describes a method for the preparation of an ethanolic extract of Trifolium, with nematicide capacity. Similarly there are several scientific publications that mention plant extracts for the control of nematodes, including of the genus Couropita and Nepeta (Pavaraj M., Bakavathiappan Ga., S. Baskaran), as well as the evaluation of some plant extracts based on their nematicidal properties against root-knot nematode, Meloidogyne incognita (J Biopest, 2012. 5 (Supplimentary): 106-110)), and Nicotiana tabacum, Syzygium aromaticum and Piper beetle plants (Wiratno; Taniwiryono, D.; Berg, J. H. J. van den; Riksen, J. A. G.; Rietjens, I.; Djiwanti, S. R.; Kammenga, J. E.; Murk, A. J. Nematicidal Activity of Plant Extracts Against the Root-Knot Nematode, Meloidogyne incognita. The Open Natural Products Journal. 2009. 2: 77-85).

Actually in the market a product can be found, QL Agri®35 (BASF SE), which corresponds to a Quillaja tree, which is a (Quillaja saponaria) extract, with nematicide capacity, especially for the control of these phytoparasites in grape and citric plantations (Giannakou I O. Efficacy of a formulated product containing Quillaja saponaria plant extracts, for the control of root-knot nematodes. Eur J Plant Pathol. 2011. 130:587-596).

On the other hand, biological origin nematicides, seek to enhance the effect on natural enemies of nematodes, using bacteria and fungi capable of killing nematodes through the capture, parasitism, opportunism or toxin production. Extracellular enzymes excreted by microorganisms play a crucial role during the infection against nematodes, with proteases, chitinases and collagenases responsible for digestion and degradation of the essential host cell proteins (Yang J, Liang L, Li J, Zhang KQ. Nematicidal enzymes from microorganisms and their applications. Appl Microbiol Biotechnol. 2013. 97: 7081-7095). Some of the characterized enzymes belong to fungus species Pochonia sp., And Paecilomyces sp., and bacteria of the Bacillus sp. species, And Pseudomonas fluorescens. They are enzymes and cellular metabolites of these species which are mixed into the product Blocker® (Biogen Agro SAC) for the control and elimination of nematodes.

However, both natural alternatives have proved less effective, more inconsistent and with a slower action, than the control achieved with the use of chemicals (Kerry B. Biological Control of nematodes: Opportunities and prospects FAO Plant Production and Protection Paper—144. Rome 1997). Although many efforts have been made and combinations of agents for controlling nematodes, to date, it has not been possible to obtain an effective nematicide composition, easy to use and friendly to the environment. Considerable shortcomings remain.

### SUMMARY OF THE INVENTION

An object of the present invention relates to a nematicidal composition which comprises as an active ingredient with nematicidal activity, monomers oligomers, derived from quinic acid and gallic acid; they also contain hydrocolloids; carbohydrates and soluble proteins and agronomically acceptable excipients. Optionally it may contain oligomers derived from monomers of caffeic acid and methylated derivatives of gallic acid, caffeic acid and quinic acid.

Such compounds are added in the composition in proportions of oligomers, of 20 to 50% by weight, hydrocolloids, as a preference galactomannans, from 0.25 to 3% by weight, consisting of starchy carbohydrates and simple sugars, between 1 and 15% by weight water-soluble proteins from 1 to 5% by weight, and sufficient agronomically acceptable excipients.

The oligomers present in the composition are derived from monomers of gallic acid, caffeic acid and quinic acid, which are in a range of concentrations from 16 to 40%, 4 to 10% and 0.1 to 0.5 wt %, respectively.

Optionally, the present nematicide composition may additionally contain saponins which have a final concentration of between 2 and 20% by weight of the final formulation.

Optionally, the present nematicide composition may also contain additionally, yeast extract in a concentration range between 1 and 5% by weight of the final formulation. Optionally, the nematicidal composition may contain a range of glucose concentrations between 1 and 5% by weight of the final formulation.

Optionally, the present nematicide composition additionally may contain live nematicide microorganisms. Preferably the live microorganism is Paecilomyces lilacinus, present in the nematicide composition in a concentration of between 10 to 1012 colony forming units (CFU) per ml.

Another objective of the present invention relates to a method for controlling plant parasitic nematodes, where the nematicide composition described is applied to cropland plants, in concentrations which can vary from 10,000 to 100,000 ppm, at doses between 10 to 100 litres per hectare applied 1 to 5 times at intervals of 5 to 150 days. This nematicide composition may be applied through drip irrigation, spray, manual or direct application in powder, granules, pellet balls or other suitable method.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 shows a table with a preferred embodiment of the nematicidal composition of the invention and possible variants thereof.

FIG. 2 shows a table with the dose and comparative results of the nematicide composition of the invention compared with commercial products used for testing mortality, mobility and hatching eggs of Meloydogyne incognita.

FIG. 3 shows a table with treatments and doses which were used for field testing of the composition of the invention.

FIG. 4 shows a graph of population dynamics of Meloidogyne sp., in the field trial on vines nematicide composition at different timing.

FIG. 5 shows a graph of population dynamics of Meloidogyne sp., in testing the nematicidal composition at higher doses, on asparagus crops.

FIG. 6 shows a graph of results of a biometric test of grapevine roots, using rhizotrons.

FIG. 7 shows a graph of results of a biometric test vines roots, using buckets.

While the system and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

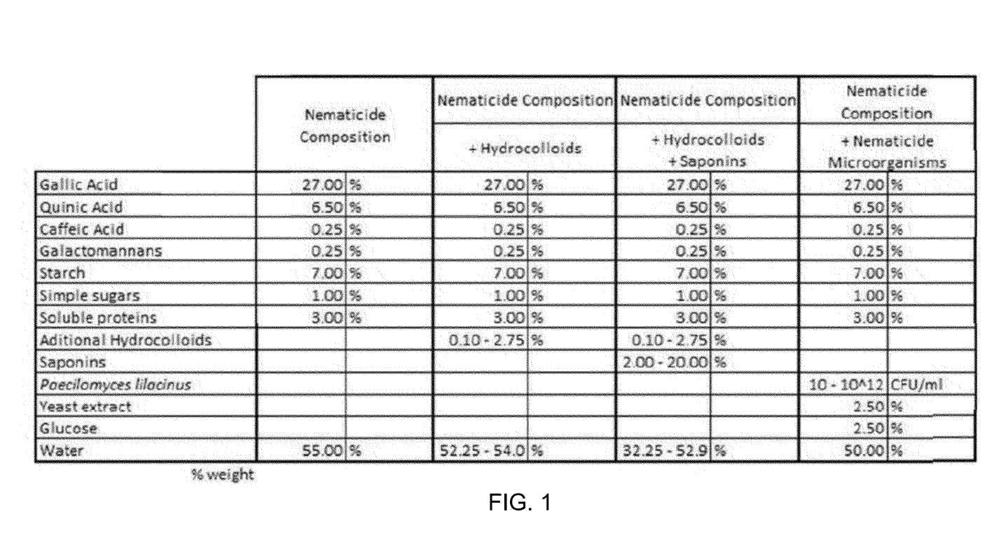

As seen in FIGS. 1-7, the present application relates to a nematicidal composition and a method for controlling plant parasitic nematodes, using such nematicidal composition which is applied to infected crop grounds, so that the composition reaches the roots of plants, where the nematodes settle preferably.

The nematicidal composition comprises oligomers formed mainly of monomers of gallic acid and quinic acid, linked through ester bonds. These acids correspond to approximately 98% of the total monomers comprising the oligomers. Additionally, such monomers may contain oligomers of caffeic acid, or methylated derivatives of gallic acid, caffeic acid and quinic acid in lower concentrations than the main compounds. Oligomer is defined as any combination in the number of monomer units of gallic acid, quinic acid, caffeic acid or any of its methylated derivatives.

Furthermore, the composition contains hydrocolloids, preferably galactomannans, starch and carbohydrates such as simple sugars, soluble proteins, and agronomically acceptable excipients.

All these compounds are preferably of natural origin and are widely distributed in leaves, bark and fruit of plants, however, they also be synthesized artificially and mixed in the corresponding proportions without changes in their final nematicidal effects. Similarly mixed compounds can be found naturally in some plant species, so that extracts can be obtained from these plants with the particular composition disclosed herein, it should also be considered as an additional embodiments of the present invention.

For the nematicidal composition to be effective, the concentrations of the components should be mixed in proportions of from 20 to 50% by weight of oligomers, this is, between 16 to 40% of gallic acid, 4 to 10% of quinic acid and 0.1 to 0.5% of caffeic acid; 0.1 to 3% by weight of hydrocolloids, 1 to 15% by weight of starch and simple sugars, and 1 5% soluble proteins and sufficient agronomically acceptable excipient.

Optionally, the nematicidal composition described above, may contain other compounds that improve its characteristics. In FIG. 1 an embodiment of the composition with the basic ingredients and some possible variants is shown. For example, it may contain hydrocolloids in addition to the main composition, which improves the adhesion of the formulation to the roots of plants, and contributes to hydration and stabilization of crops and improvements in its root system hydrocolloids. Hydrocolloids included in the composition, additionally will range from 0.1 to 3% by weight of the final formulation. Furthermore, the nematicidal composition may additionally contain saponins, compounds known for their toxic properties. The addition of saponin to the composition will range from 2 to 20% by weight of the final formulation. Another possibility envisaged, is the addition of live microorganisms to the nematicidal composition. They help to develop or enhance the plant nematicide formulation. Paecilomyces lilacinus are selected preferably, a fungus known in the prior art for their nematicidal properties, but any other microorganism could be used of the same characteristics. The addition of these microorganisms is in the range of 10-10¹² CFU/ml. For the adequate survival of the microorganism, it should be additionally added to the composition; glucose and yeast extract, each in the range of 1 to 5% by weight of the final formulation.

A second objective of the present invention is a method for the control of parasitic plant nematodes in crops in which the composition applied is a nematicide in any of its variants. The composition can be used on any ground or soil, such as in massive culture lands, glasshouses, pots and gardens. The administration form of the composition may be a dry substance, such as powders, capsules, pellet, balls or tablets, as a liquid or as a solution or as a foam, gel, emulsion or any other form acceptable in agriculture. The final formulations may also contain agronomically acceptable excipients, such as disintegrating and/or binding, wetting agents, surfactants and stabilizers.

The concentration and dosage of the composition is calculated depending on the amount of phytopathogenic nematodes found in the crops, and to the type of soil where these are found. In a preferred embodiment, the concentration range is between 10,000 to 100,000 ppm, and the dose range between 10-100 litres per hectare of crop, at intervals of 1 to 5 times every 5 to 150 days. The application is made by using technically advanced methods such as through drip irrigation systems or manual irrigation systems, in the case of large areas, but can also be applied on the soil or on the plants, by spraying, dusting, sprinkling or the like.

The present invention provides an alternative to the commonly used chemical nematicides, with naturally occurring compounds for the control of phytopathogenic nematodes, more efficient and better consistency, than the currently available products on the market. The nematicidal composition is capable of controlling a wide range of species and genus of nematodes in various crops and soils. Additionally, the composition of the invention surprisingly showed an activation and promotion of the root growth of plants, a protective effect on new roots was also observed, facilitating the healthy growth and development of the plant and its fruit.

Below embodiments of the invention have been included for the purpose of illustrating the invention. The preferred embodiments and comparative examples are presented, but in no case they should be considered that they restrict the scope of the invention which is only limited by the content of the claims appended hereto.

### EXAMPLES

### Example 1

### In vitro Assays of the Nematecide Composition

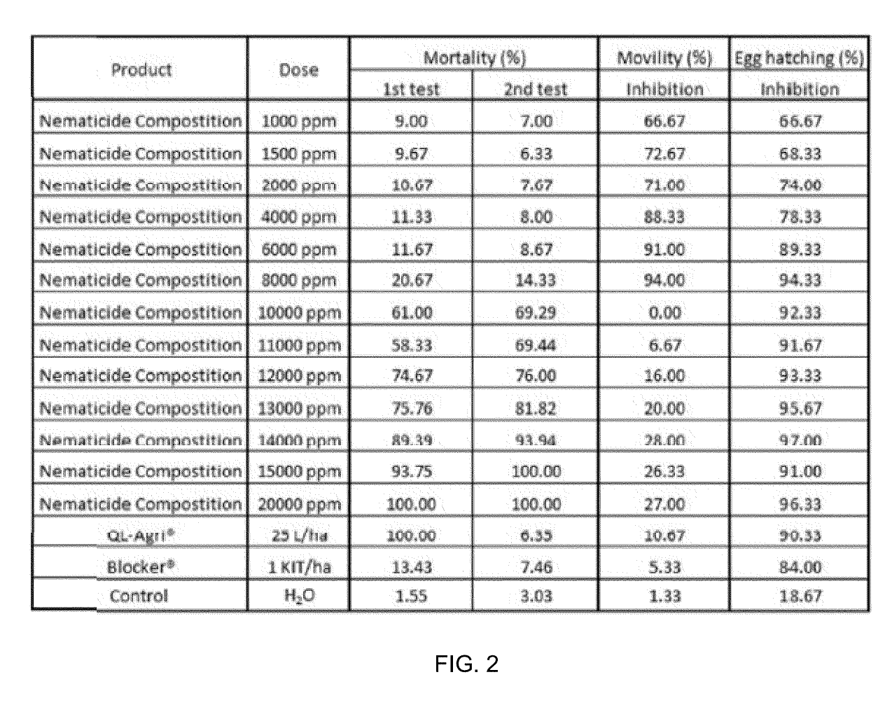

In vitro assays of nematicide composition of the present invention in different concentrations and comparative tests in comparison with the natural product QL-Agri® were performed, and with the biological origin product of Blocker®, both at the recommended doses indicated in their sales brochures in relation to Meloydogine incognita nematodes. The mortality was measured on 100 nematodes in their juvenile stage 2 of M. incognita, after being exposed during 48 hours to different treatments. The number of nematodes after 8 hours of post-treatment was analyzed to evaluate the recovery of nematodes. Mobility was measured on 100 individuals of nematodes in the juvenile stage 2 of M. incognita, after being exposed during 72 hours, to different treatments. The percentage of affected individuals was determined in relation to the motion inhibition percentage. Furthermore hatching rate was measured on 100 eggs of M. incognita after 96 hours of exposure to the different treatments. All the assays were performed 3 times in laboratory conditions. A control testing sample, corresponding to water was used as control. FIG. 2 shows a table with the doses used in each of the tests, and the results of mortality, mobility and egg hatching tests. The results of these test show that the nematicidal composition is highly efficient in doses above 10,000 ppm and produces increased mortality, decreased mobility and hatching, than the commercial products currently available in the market.

### Example No. 2

### Field Assays of the Nematicide Composition and Variables of it

Assays were performed in the field, of the nematicide composition of the present invention in doses of 15,000 to 20,000 ppm equivalent to 13.2 L/ha and 17.6 L/ha ppm, respectively, and comparative tests with natural product QL-Agri® and with the product of biological origin, Blocker®, both at the recommended doses indicated in their sales brochures. Furthermore, the variant of the nematicidal composition with additional hydrocolloids, additional to the hydrocolloids contained in the original formulation, was evaluated. In particular for this assay, Kelpak® commercial hydrocolloid was added, in a concentration of 4 L/ha to the nematicidal composition, and this mixture was studied at the same doses as the base nematicide composition, i.e. 15,000 and 20,000 ppm equivalent to 13.2 L/ha and 17.6 L/ha, respectively.

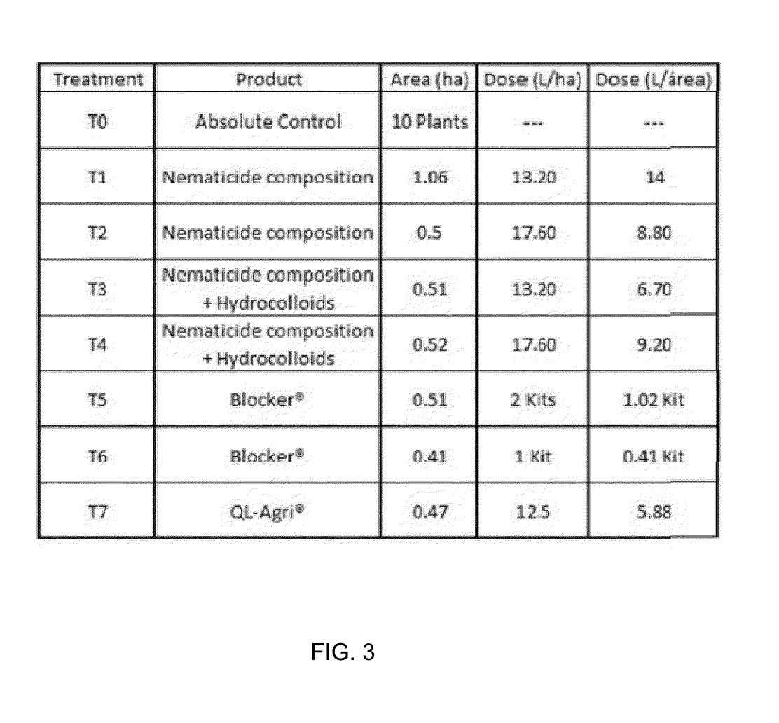

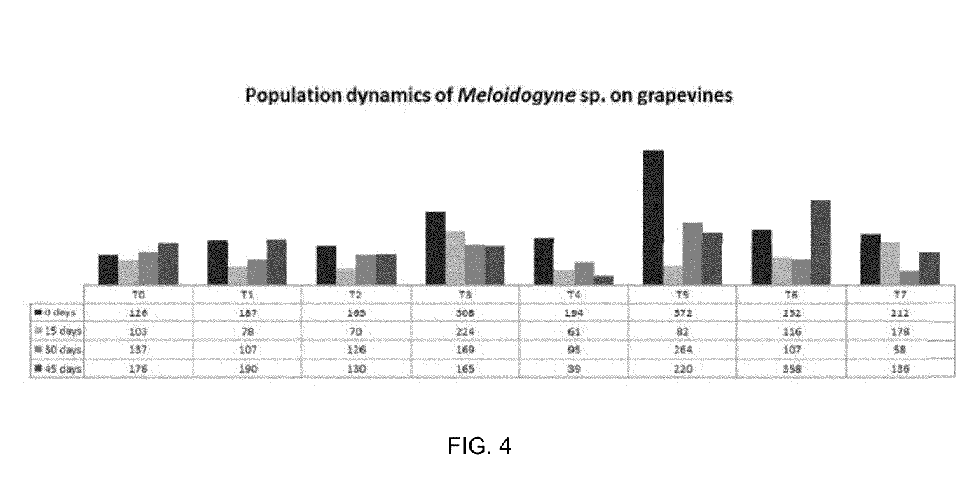

The tests were performed in six different fields with different soil conditions in culture plantations of vines, avocado and citrus. During 60 days 4 times the different treatments were applied, every 15 days, and 4 samples were taken during said period of time. In an initial test on vines, it was found that, the main pathogenic nematodes were Meloidogyne sp., and Rotylenchulus sp. FIG. 3 shows a table with the description of the treatments applied in this test. FIG. 4 shows a graph of the results of the population dynamics of Meloidogyne sp., in the field testing of the vines culture plantation.

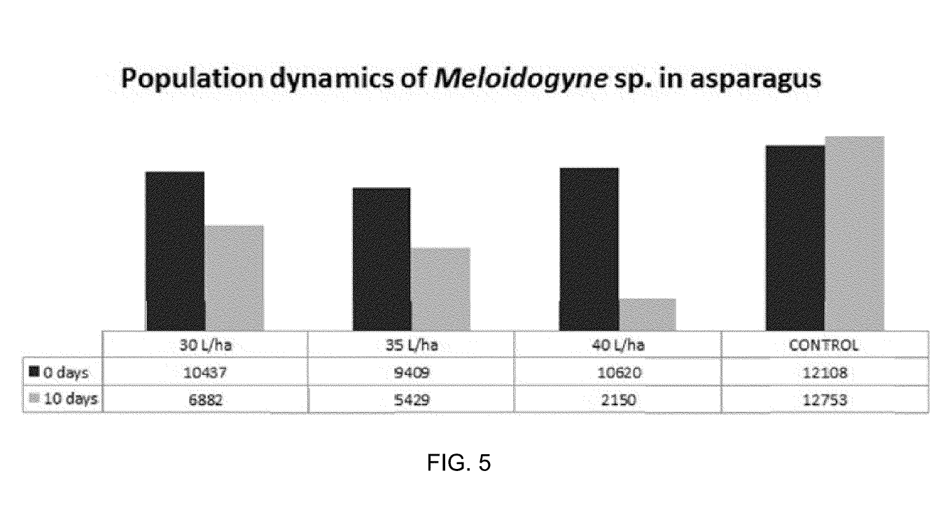

In a second field assay, the effect of higher doses of the base nematicidal composition, on asparagus crops were analyzed. Initially, it was found on the soil of said crops, a high concentration of Meloidogyne sp. nematode, which decreased by applying higher doses of the nematicidal composition. It was tested at doses of 30 L/ha, 35 L/ha and 40 L/ha. FIG. 5, shows a graph of the dynamics of the nematode populations treatment at different concentrations, each application made on day 0 and then at day 10.

The results show that both the base nematicide composition based on different concentrations, as the nematicide composition containing additional hydrocolloids, have a negative effect on the nematode population and its effect is better than commercial alternatives evaluated.

### Example No. 3

### Root Growth Analysis

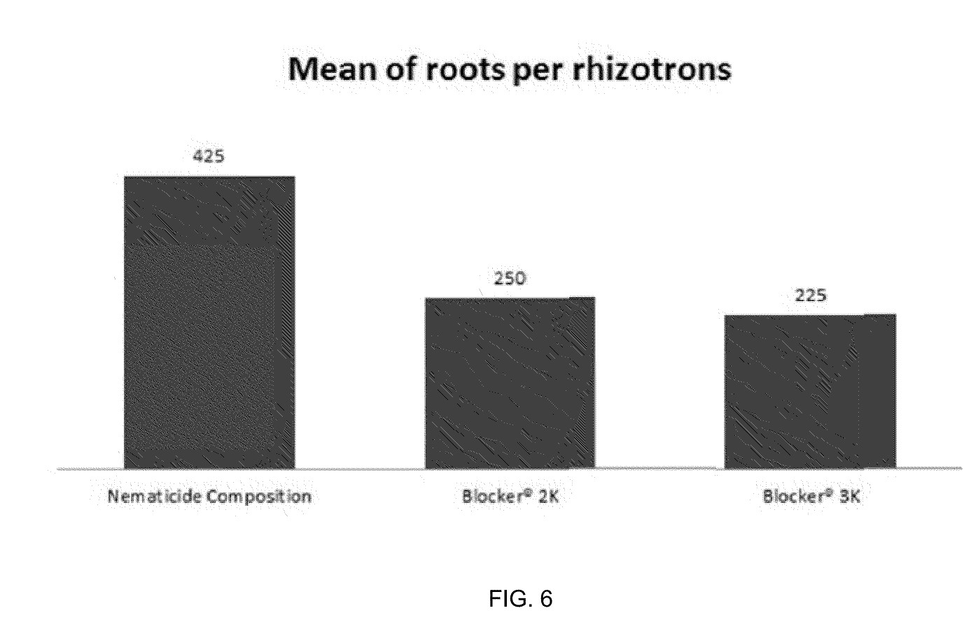

A biometric study was realized, using rhizotrons and cubes. The rhizotrons are wooden compartments with a glass bottom of 50×60 cm and 6 mm thick, installed next to the roots, in order to evaluate the root growth. These rhizotrons are installed at 20-25 cm of the neck of the plant, at a depth of 60 cm from ground level. The evaluation was performed 15 and 30 days after application of treatments. The comparison was made applying as a treatment nematicide composition versus the commercial products Blocker® 2K (2 kits) and Blocker® 3K (3 kits). The results of this analysis are shown in FIG. 6.

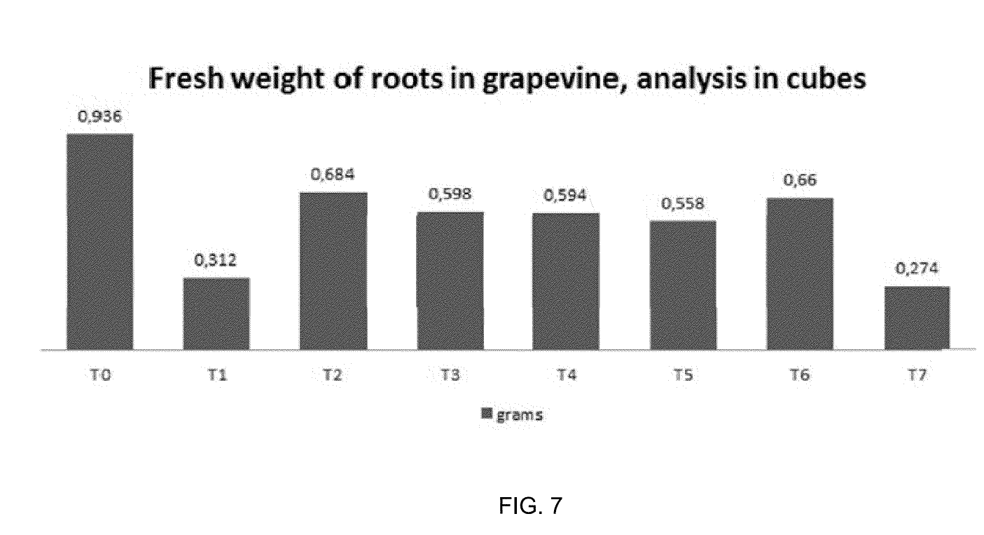

In turn, the cubes are built with screens 35×35 cm, assembled as cubes which were installed between plants in the case of vines, at a depth of 40 cm from ground level. The evaluation was performed two months after the treatment was applied. In this case, the growth of roots of the vines treated with nematicide composition at doses of 13.2 and 17.6 L/ha, a nematicidal composition with additional hydrocolloids was applied in doses of 13.2 and 17.6 L/ha, was compared with the commercial products, Blocker® 1K, 2K Blocker® (2 kits) and QL-Agri. The results of this assay are shown in FIG. 7 in fresh root weight after the treatments.

The results of this study show that both the nematicide composition as the nematicide composition with additional hydrocolloids, improve plant rooting in comparison to other available commercial products, like Blocker® and QL-Agri®.

The particular embodiments and examples disclosed above are illustrative only, as the application may be modified and practiced in different but equivalent manners apparent to those skilled in the art having the benefit of the teachings herein. It is therefore evident that the particular embodiments and steps disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A nematicidal composition CHARACTERIZED in that it comprises:
quinic acid and gallic acid oligomers;
hydrocolloids;
carbohydrates;
water-soluble proteins; and
agronomically acceptable excipients.

2. The nematicidal composition of claim 1 CHARACTERIZED in that it optionally contains oligomers of caffeic acid and methylated derivatives of gallic acid, caffeic acid and quinic acid.

3. The nematicidal composition of claim 1, CHARACTERIZED in that the oligomers concentration is from 20 to 50% by weight of the final formulation.

4. The nematicidal composition of claim 1, CHARACTERIZED in that the gallic acid concentration is between 16-40% by weight, quinic acid is between 4-10% by weight and caffeic acid is between 0.1 to 0.5% by weight.

5. The nematicidal composition of claim 1, CHARACTERIZED in that the total hydrocolloid concentration is between 0.25 and 3% by weight of the final formulation.

6. The nematicidal composition of claim 5, CHARACTERIZED in that the hydrocolloids are preferably galactomannans.

7. The nematicidal composition of claim 1, CHARACTERIZED in that the carbohydrates are starch and simple sugars, the concentration is between 1 and 15% by weight of the final formulation.

8. The nematicidal composition of claim 1, CHARACTERIZED in that the water-soluble protein concentration is between 0.5 and 8% by weight of the final formulation.

9. The nematicidal composition of claim 1, CHARACTERIZED in that it optionally comprises saponins.

10. The nematicidal composition of claim 8, CHARACTERIZED in that the saponin concentration is between 2-20% of the final formulation.

11. The nematicidal composition of claim 1, CHARACTERIZED in that it optionally comprises yeast extract at a concentration between 1 and 5% by weight of the final formulation.

12. The nematicidal composition of claim 1, CHARACTERIZED in that it optionally comprises glucose.

13. The nematicidal composition of claim 12, CHARACTERIZED in that the glucose concentration is between 1 and 5% by weight of the final formulation.

14. The nematicidal composition of claim 1, CHARACTERIZED in that it optionally comprises living nematicides organisms.

15. The nematicidal composition of claim 14, CHARACTERIZED in that the concentration of living nematicide organisms is between 10-10¹² CFU/ml.

16. The nematicidal composition of claims 14, CHARACTERIZED in that the living nematicide organisms is Paecilomyces lilacinus.

17. A method for controlling plant parasitic nematodes, CHARACTERIZED in that it comprises:
providing a nematicide composition comprising quinic acid and gallic acid oligomers, hydrocolloids, carbohydrates, water-soluble proteins and agronomically acceptable excipients; and
applying said composition to crop soil.

18. The method of claim 17, CHARACTERIZED in that the composition is applied to the crop soil at a dosage between 10,000 to 100,000 ppm.

19. The method of claim 17, CHARACTERIZED in that the composition is applied at a dose between 10-100 L/ha of 1 to 5 times at intervals ranging from 5 to 150 days.

20. The method of claim 17, CHARACTERIZED in that the composition is applied using a technique selected from the group consisting of drip irrigation, spraying, atomizing, manual watering, and direct application in the form of powder, granules, capsules and ball shape pellets.
